(19) United States
(12) Patent Application Publication
(10) Pub. No.: US 2008/0039635 A1
(43) Pub. Date: Feb. 14, 2008

(54) Method for the Production of a Compound, Comprising a Free Hydroxyl Group and a Hydroxyl Group Which is Protected by an Ester Function by Enzymatic Reaction

(75) Inventors: Laurent Garel, Lyon (FR); Mirjana Gelo-Pujic, Serezin Du Rhone (FR); Thierry Schlama, Buschwiller (FR)

Correspondence Address: BUCHANAN, INGERSOLL & ROONEY PC, POST OFFICE BOX 1404, ALEXANDRIA, VA, 22313-1404, US

(73) Assignee: RHODIA-CHIMIE, Aubervilliers Cedex (FR)

(21) Appl. No.: 10/576,773

(22) PCT Filed: Oct. 19, 2004
(86) PCT No.: PCT/FR04/02665

§ 371 Date: Apr. 25, 2007

(30) Foreign Application Priority Data
Oct. 22, 2003 (FR) 0312329

(51) Int. Cl. C07D 205/00; C07D 305/00; C07D 331/04

(52) U.S. Cl. 548/952; 549/510; 549/88

(57) ABSTRACT

The invention relates to a method for the production of a compound comprising a free hydroxyl group and a hydroxyl group which is protected by an ester function by enzymatic reaction, using a lipase EC 3.1.1.3. The invention also relates to the use of the resultant compound as an intermediate for the production of medicaments and pharmaceutical products.

The present invention relates to a process for the manufacture of a compound comprising a free hydroxyl group and a hydroxyl group protected by an ester functional group by enzymatic reaction using a lipase of the class EC 3.1.1.3. The present invention also relates to the use of this compound as intermediate in the manufacture of medicaments and pharmaceuticals.

Chiral synthons comprising a free hydroxyl group and a hydroxyl group protected by an ester functional group are particularly advantageous in the asymmetric synthesis of pharmaceuticals. Thus, chiral synthons of 1-acetoxy-4-hydroxycyclopent-1-ene type are particularly used as precursors of prostaglandins, prostacyclins and thromboxanes.

There exist in particular in the prior art several processes developed for the preparation of enantiomerically pure S and/or R monoacetate compounds by enzymatic catalysis of the monoacylation reaction by enzymes mainly of animal origin. For example, pancreatin, an enzyme originating from the pig pancreas, catalyses the monoacetylation reaction of 1,4-dihydroxycyclopent-2-ene in the manufacture of enantiomerically pure S monoacetate compounds.

However, for reasons of medical safety, it is the wish of national regulatory authorities eventually to end the use of products of animal origin in the manufacture of medicaments and pharmaceuticals. There thus exists a need to develop processes for the manufacture of pharmaceutical chiral intermediates using enzymes which are not of animal origin.

Furthermore, there also exists a need to bring to the fore effective enzymes, in particular effective at low amounts, which make it possible to obtain good selectivity for the chiral synthons desired.

There exist in the prior art processes for the manufacture of enantiomerically pure chiral synthons using enzymes of plant or microbial origin. However, the majority of these processes described are difficult to transfer to the industrial scale. It is thus very advantageous to have available an industrial process which makes possible access to the enantiomerically pure chiral synthons desired.

The present invention relates to a novel process for the manufacture of chiral synthons comprising a free hydroxyl group and a hydroxyl group protected by an ester functional group using a lipase originating from the Gram-negative bacteria Alcaligenes spp.

The manufacturing process comprises an enzymatic transesterification of a diol compound by a lipase and an acylating agent, for example, in the following way:

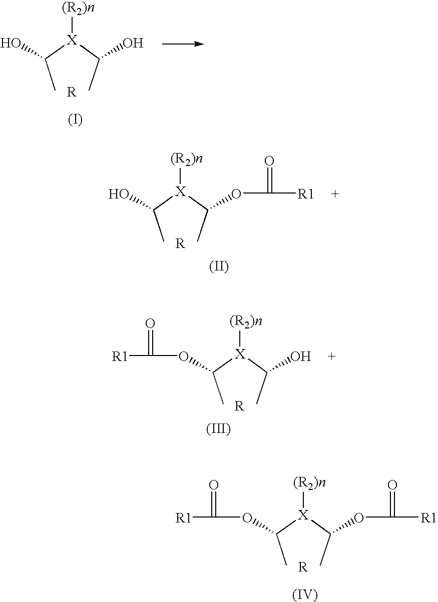

The use of the lipase thus makes it possible to obtain a transesterification reaction of the compound (I) with a better performance than that obtained with an enzyme of animal origin, with markedly smaller amounts.

Starting from the compound of formula (II) of the invention, it is possible to manufacture medicaments and pharmaceuticals using this compound as intermediate.

A first subject matter of the present invention is a process for the manufacture of a compound of formula (II):

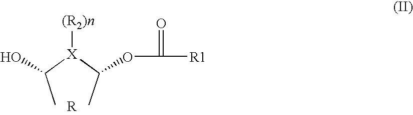

in which:

R is a covalent bond or a hydrocarbon chain comprising from 1 to 10 carbon atoms, preferably from 1 to 5 carbon atoms;

R¹ is a hydrocarbon group comprising from 1 to 10 carbon atoms, preferably from 1 to 6 carbon atoms;

R² corresponds to a hydrogen atom and n is an integer between 0 and 2, that is to say 0, 1 or 2;

X is an atom chosen from the group consisting of carbon, nitrogen, oxygen and sulfur, preferably carbon; comprising at least the following stages:

a) at least one compound of formula (I):

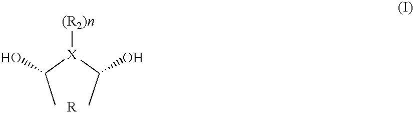

is reacted with an acylating agent in an organic solvent in the presence of a lipase of the class EC 3.1.1.3 from the genus Alcaligenes spp., so as to form the compound of formula (II);

b) the compound of formula (II) is isolated.

Another subject matter of the present invention is a compound of formula (II) capable of being obtained by the process as described above. Another subject matter of the present invention is a composition capable of being obtained by the process as described above.

The lipases used according to the invention are lipases of the class EC 3.1.1.3 from the genus Alcaligenes spp. The genus Alcaligenes spp. of the family Alcaligenaceae comprises) several species, such as, for example: Alcaligenes aestus, Alcaligenes aquamarinus, Alcaligenes cupidus, Alcaligenes defragrans, Alcaligenes denitrificans, Alcaligenes eutrophus, Alcaligenes faecalis, Alcaligenes latus, Alcaligenes pacificus, Alcaligenes paradoxus, Alcaligenes piechaudii, Alcaligenes ruhlandii, Alcaligenes venustus or Alcaligenes xylosoxidans.

Use may be made, according to the process of the invention, of different lipases in accordance with the invention.

The lipases of the invention can be obtained from culturing Alcaligenes spp. The culturing conditions can vary according to the type of strain used. It is recommended to choose these conditions so as to produce the lipases in the most advantageous manner possible. The culturing temperature is generally between 5 and 50° C. The culturing period is generally between 1 and 10 days.

The Alcaligenes spp. lipases can be recovered in various ways well known to a person skilled in the art. It is, for example, possible to separate the bacteria and the culture medium, in particular by centrifuging or filtering, and subsequently to purify the lipases. For example, the lipases can be purified by precipitation, lyophilization, ion-exchange chromato-graphy, immunoaffinity using specific mono- or polyclonal antibodies, and/or dialysis. It is also possible to collect the Alcaligenes spp. lipases by destroying the bacteria, for example by sonication and recovery of the ground material, or by enzymatic lysis of the cell walls. The lipases of the invention can in particular be purified from Alcaligenes spp. strains by addition of salts, for example using ammonium sulfate, passing through an ion-exchange chromatograph and subsequent gel filtration.

It is clearly understood that natural, mutated synthetic, chimeric and/or recombinant lipases originating from Alcaligenes spp. can be used in the process according to the invention, insofar as their transesterification activity with regard to the substrates of the invention is retained, indeed even improved. Thus, the present invention also relates to Alcaligenes spp. mutated lipases expressed by other microorganisms or produced by chemical synthesis.

Preferably, the Alcaligenes spp. lipases used according to the invention make it possible to obtain the following parameters:

enantiomeric excess of compound (II) of greater than or equal to 50%, preferably of greater than or equal to 70%, particularly of greater than or equal to 90%, very particularly equal to 100%;

selectivity for compounds (II) and (III) of greater than or equal to 2, preferably of greater than or equal to 2.5, particularly of greater than or equal to 3.3;

yield of compound (II) of greater than or equal to 40%, preferably of greater than or equal to 50%, particularly of greater than or equal to 75%; and

degree of conversion of the compound (I) of greater than or equal to 70%, preferably of greater than or equal to 90%, particularly of greater than or equal to 95%.

The following test can be carried out in order to determine these parameters: 4.21 g (0.0421 mol) of 1,3-dihydroxycyclopent-2-ene (corresponding to compound (I)), hereinafter referred to as diol, are introduced with stirring into 30 ml of acetone (23.7 g) at ambient temperature (24-25° C.) in a 100 ml reactor. After dissolving the 1,3-dihydroxycyclopent-2-ene in the acetone, 18.08 g (0.215 mol; 5 molar equivalents with respect to the diol) of vinyl acetate are added, followed by 420 μl (10% by weight with respect to the diol) of demineralized water. The temperature of the medium is then set at 5° C. 5% by weight of lipase enzyme of the class EC 3.1.1.3 from Alcaligenes spp. with respect to the diol are added. After 12 hours, a 0.5 ml sample of reaction medium is withdrawn and centrifuged. 200 μl are withdrawn and diluted in 800 μl of acetone before injection in chiral gas chromatography. The characteristics mentioned above are subsequently measured as explained in the experimental part.

This test allows a person skilled in the art to determine the lipases of the class EC 3.1.1.3 originating from the genus Alcaligenes spp. and/or their fragments which have an activity in accordance with the invention.

Preferably, the lipases used according to the process of the invention exhibit an amino acid sequence having a percentage homology of greater than or equal to 80%, in particular 90%, preferably 95%, more preferably 100%, with the amino acid sequence of the QL lipase from Alcaligenes sp. PL-266, registered under the number FERM-P No. 3187, the protein having a transesterification activity with regard to the substrates of the invention. The lipases used according to the invention can originate from nucleotide sequences exhibiting a percentage homology of greater than or equal to 80%, in particular 90%, preferably 95%, more preferably 100%, with the nucleotide sequence of the gene of the QL lipase from Alcaligenes sp. PL-266, registered under the number FERM-P No. 3187, the lipase obtained having a transesterification activity with regard to the substrates of the invention.

It is understood that the term “homology” refers to the perfect resemblance or identity between the amino acids compared but also to the imperfect resemblance, which is described as “similarity”. The amino acid sequence can differ from the reference sequence by substitution, deletion and/or insertion of one or more amino acids, preferably of a reduced number of amino acids, in particular by substitution of natural amino acids by unnatural amino acids or pseudoamino acids, at positions such that these modifications do not significantly affect the biological activity of the protein. The homology is generally determined using a sequence analysis software (for example, Sequence Analysis Software Package of the Genetics Computer Group, University of Wisconsin Biotechnology Center, 1710 University Avenue, Madison, Wis. 53705; or BLAST software of the National Center for Biotechnology Information, US National Library of Medicine, 8600 Rockville Pike, Bethesda, Md. 20894).

The amino acid sequences of the natural, mutated synthetic, chimeric and/or recombinant lipases can have the same length as the reference sequences.

It is understood, according to the invention, that the lipases of the class EC 3.1.1.3 can originate from the bacteria of the genus Alcaligenes spp. However, for example in the context of an industrial process for the manufacture of these lipases, it is possible for the latter to be produced by host cells or by chemical processes.

The nucleotide sequences that result in the synthesis of the natural, mutated synthetic, chimeric and/or recombinant lipases can be inserted into vectors that replicate autonomously in the chosen host, or integrative vectors. Such vectors will be prepared according to the methods commonly used by a person skilled in the art, and the clones resulting therefrom can be introduced into an appropriate host by standard methods, such as, for example, electroporation or transformation with calcium chloride, polyethylene glycol or fusion of protoplasts. The signals controlling the expression, or the overexpression, of the nucleotide sequences (promoters, activators, termination sequences, and the like) are chosen according to the cellular host used. The host cells can be transiently or stably transfected with these expression vectors. These cells can be obtained by introduction into prokaryotic or eukaryotic host cells. Examples of host cells include in particular mammalian cells, such as 293 or CMV (cytomegalovirus) cells, insect cells, such as cells derived from Spodoptera frugiperda ovary or cells from Drosophila melanogaster embryos, bacteria, such as E. coli or B. subtilis, and yeast strains, such as Saccharomyces cerevisiae.

The lipases of the invention can also be produced by chemical synthesis. To this end, recourse may be had to any method well known to a person skilled in the art. The peptide of the invention can, for example, be synthesized by the techniques of synthetic chemistry, such as synthesis of Merrifield type, which is advantageous for reasons of purity, antigene specificity and absence of undesired byproducts and for its ease of production. The chemical synthesis makes it possible in particular to produce nucleotide sequences or amino acid sequences optionally comprising substitutions, deletions and/or insertions with respect to a reference sequence.

Direct use may also be made in the process of whole cells of Alcaligenes spp., optionally recombined so as to overexpress the lipases at a satisfactory level. To this end, it is possible to insert, into the genome of the microorganism, one or more expression cassettes comprising the nucleotide sequence expressing the lipases of the invention, under the control of one or more element(s) allowing its expression or the regulation of its expression, such as, in particular, transcription promoters, activators and/or terminators.

By way of example, use may in particular be made of the QL lipase from Alcaligenes sp. PL-266, registered under the number FERM-P No. 3187 (also referred to as QLM), for example mentioned in patent JP 58-36953, or the PL lipase from Alcaligenes sp. PL-679, registered under the number FERM-P No. 3783, but also ATCC 31371 and DSM 1239, for example mentioned in JP 60-15312.

The lipase may or may not be immobilized on an appropriate solid support. The solid support can be chosen from the group consisting of DEAE cellulose, DEAE sepharose, diatomaceous earth, silica, alumina, polypropylene, ceramic particles and/or their mixtures.

Use may be in particular be made, as lipases of the class EC 3.1.1.3 from Alcaligenes spp., of Chirazyme™ L-10, sold by Roche, or the QL (or QLM), QLC, QLG, PL, PLC and PLG lipases sold by Meito-Sangyo. The PLC and PLG lipases correspond respectively to the PL lipase immobilized on diatomaceous earth and on granules of diatomaceous earths. The QLC and QLG lipases correspond respectively to the QL lipase immobilized on diatomaceous earth and on granules of diatomaceous earths.

Numerous substrates of formula (I) can be monoacylated according to the process of the present invention.

The R group can be a covalent bond or a saturated or unsaturated, linear or branched and aliphatic, cyclic and/or aromatic hydrocarbon chain comprising from 1 to 10 carbon atoms, preferably from 1 to 5 carbon atoms, which can comprise and/or form one or more optionally aromatic rings. This hydrocarbon chain can optionally comprise one or more heteroatoms chosen from the group consisting of carbon, nitrogen, phosphorus, oxygen, silicon and sulfur.

If R′ is a covalent bond, the compound of formula (I) will be a compound derived from cyclopropane.

Preferably, R is a hydrocarbon chain comprising at least one unsaturation. R can be a hydrocarbon chain comprising one or more aromatic or nonaromatic ring(s).

For example, if the compound of formula (I) is cis-4-cyclopentene-1,3-diol, the R group corresponds to an unsaturated hydrocarbon chain comprising 2 carbon atoms.

The (R² )ₙ group depends on the valency of the X atom. For example, if X is a carbon atom, R²=H and n=2. If X is an oxygen or sulfur atom, R²=H and n=0. Likewise, if X is a nitrogen atom, R²=H and n=1.

Preferably, the compound of formula (I) is chosen from the group consisting of the compounds of formula (V), (VI) and/or (VII):

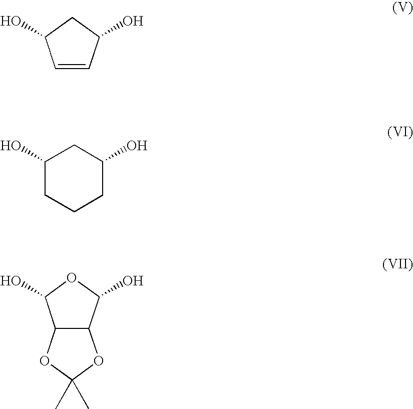

The preferred substrate according to the invention is cis-4-cyclopentene-1,3-diol. The compound of formula (II) obtained from this substrate according to the process of the invention is (1R, 4S)-4-acetoxy-cyclopent-2-en-1-ol.

The isolated lipase can be employed in aqueous solution, optionally buffered, in organic solvents, in single-phase or two-phase solution.

Various types of organic solvents can be used according to the present invention. The solvent is by definition capable of at least partially dissolving the substrate, such as the compound of formula (I). Preference is given in particular to a water-miscible organic solvent. The organic solvent can be an aliphatic, cyclic or aromatic hydrocarbon compound optionally comprising chlorinated, nitrogenous, acid, ketone, nitrile, aldehyde and/or ester functional groups.

The organic solvent is preferably chosen from the group consisting of: ketones, such as acetone, methyl ethyl ketone, cyclohexanone, cyclopentanone and methyl isobutyl ketone (MIBK); ethers, such as methyl tert-butyl ether (MTBE) and tetrahydrofuran (THF); nitrites, such as acetonitrile; and aromatic compounds, such as toluene.

Preferably, the solvent is compatible with the lipase of the invention, that is to say that it does not decompose the protein and/or that it does not reduce its biological activity with regard to the process of the invention by more than 30%, measured, for example, with respect to the yield, the enantiomeric excess of compound (II), the degree of conversion of the compound (I) and/or the selectivity.

In addition to the organic solvent, the reaction medium, in particular that of stage a), can comprise water, for example from 0.1 to 30% by weight of water with respect to the weight of the compound of formula (I), preferably from .5 to 20% by weight of water, in particular from 5 to 30% by weight of water.

The term “acylating agent” is understood to mean a compound capable of reacting with a hydroxyl functional group of the compound (I) so as to protect the latter via an ester functional group.

The acylating agent can in particular be an ester, an anhydride or a carbonate.

The acylating agent can be a compound of formula (VIII):

R¹-COO-R³ (VIII)

in which:
R¹ is defined above; and
R³ is an optionally linear, cyclic, aromatic, branched, saturated and/or unsaturated hydrocarbon group comprising from 1 to 10 carbon atoms and optionally comprising one or more heteroatoms, such as oxygen, nitrogen, sulfur, phosphorus or chlorine. R³ is preferably an alkyl group comprising from 1 to 6 carbon atoms, optionally substituted by one or more fluorine atoms, or an alkenyl group comprising from 2 to 6 carbon atoms.

R¹ can be an optionally linear, cyclic, aromatic and/or branched hydrocarbon group comprising from 1 to 10 carbon atoms, preferably from 1 to 6 carbon atoms, and optionally comprising one or more heteroatoms, such as oxygen, nitrogen, sulfur, phosphorus or chlorine. R¹ can be chosen from the group consisting of methyl, ethyl, propyl, phenyl and isopropyl.

This acylating agent can be chosen from the group consisting of acetates, benzoates and isobutyrates.

The acylating agent is preferably chosen from the group consisting of vinyl acetate, ethyl acetate, isopropyl acetate, 2,2,2-trifluoroethyl acetate and isopropenyl acetate.

The acylating agent can also be used as organic solvent.

According to the process of the invention, the proportion of acylating agent is preferably greater than 1 molar equivalent with respect to the compound of formula (I), preferably of between 2 and 6 molar equivalents, more preferably of between 1 and 10 molar equivalents.

The reaction medium can be obtained by mixing the compound of formula (I) and optionally the acylating agent with the solvent and subsequently adding the lipase of the invention. It is also possible to obtain the reaction medium by successive addition of the following products: compound of formula (I), lipase, solvent and, finally, acylating agent.

The optimum concentrations of enzyme can be determined for each substrate and can vary within fairly wide proportions. According to the process of the invention, the proportion of lipase can be between 0.1 and 30% by weight with respect to the weight of the compound of formula (I), preferably from 0.1 to 20% by weight, particularly from 0.5 to 10% by weight.

The enzymatic catalysis reaction of stage a) is preferably carried out at a temperature of between −5 and 40° C., preferably between 1 and 15° C.

For each substrate, it is possible to determine in advance and/or to continuously monitor the duration of the enzymatic reaction as a function of the compound of formula (I) used. A person skilled in the art is fully in a position to easily determine the optimum conditions of duration for a given substrate. This can be carried out by taking regular samples of the reaction medium on which the change in the enantiomeric excess and the degree of conversion are evaluated. The duration of the enzymatic reaction of stage a) is generally between 1 and 24 hours, preferably between 4 and 16 hours.

The enzymatic reaction is carried out in an appropriate reactor optionally equipped with suitable stirring or mixing means.

The enzymatic reaction can in particular be halted by any appropriate chemical means, such as by addition of a solvent, addition of base or of acid, addition of detergents and/or addition of salts; or appropriate physical means, such as, for example, by freezing, centrifuging, heating and/or filtering.

According to stage b), the compound of formula (II) can be isolated in various ways known to a person skilled in the art, such as, for example, by purification by filtration, extraction, distillation, crystallization, column chromatography and/or centrifuging. It is possible in particular, at the end of the reaction, to carry out a filtration, followed by one or more distillations and by a crystallization, before filtration.

Another subject matter of the present invention is the use of a compound of formula (II) obtained by a manufacturing process as defined above as intermediate in the manufacture of a medicament or of a pharmaceutical, such as prostaglandins, prostacyclins and/or thromboxanes. It is possible, starting from the compound of formula (II) of the invention, to manufacture medicaments and pharmaceuticals using this compound as intermediate. Thus, for example, the use of (1R, 4S)-4-acetoxycyclopent-2-en-1-ol, corresponding to a compound of formula (II), is used for the synthesis of pharmaceuticals, as mentioned in patent WO 95/26729 and the following publications: J. Stjenschantz et al., Drugs of the Future, 1992, 17, 691; Noyori et al., Angew. Chem. Int. Ed., 1984, 23, 847; Kaumen et al., J. Chem. Soc., Chem. Comm., 1986, 1298; Kondo et al., Angew. Chem. Int. Ed., 1975, 14, 103; Tomoskozi et al., Tetrahedron Lett., 1976, 4639.

Examples of the practical implementation of the invention are given below. The following examples illustrate the invention without, however, limiting it.

### EXAMPLE 1

### Materials Used:

Lipase from Alcaligenes sp.: GLG, QLC or QL, sold by Meito-Sangyo, or Chirazyme L10™, sold by Roche (hereinafter referred to as L10);

Pancreatin lipase: porcine pancreatin, sold by Sigma;

Diol substrate: cis-4-cyclopentene-1,3-diol, sold by Fluka (compound of formula (I)).

4.21 g (0.042 mol) of 1,3-dihydroxycyclopent-2-ene are introduced with stirring into 30 ml of acetone (23.7 g) at ambient temperature (24-25° C.) in a 100 ml reactor.

After dissolution of the diol in the acetone, 18.08 g of vinyl acetate (0.21 mol; 5 molar equivalents with respect to the diol) are added, followed by 420 μl (10% by weight with respect to the diol) of demineralized water. The temperature of the medium is then set at 5° C.

100% by weight of pancreatin enzyme with respect to the diol or a predetermined percentage by weight of lipase enzyme from Alcaligenes sp. with respect to the diol are added. After 6.5 hours, 0.5 ml of reaction medium is withdrawn and centrifuged. 200 μl are withdrawn and diluted in 800 μl of acetone before injection in chiral gas chromatography.

The remainder of the reaction medium is filtered, so as to separate the lipase; the cake is washed with approximately 3 g of acetone. The filtrate, to which approximately 8 g of TMBE (tert-butyl methyl ether) have been added, is subsequently distilled under vacuum, so as to remove the acetone and the vinyl acetate. At the end of this first devolatilization, approximately 15 g of TMBE and active charcoal are added. The reaction medium is stirred and filtered through Clarsel. The filtrate is devolatilized. Heptane is subsequently added to crystallize the desired product and the temperature is reduced from 28 to 8° C. Crystallization is observed to begin at approximately 16° C. The desired compound, which exists in the form of white crystals, is subsequently filtered off. The crystals, comprising the R monoacetate (compound III) and the S monoacetate (compound II), are dried at ambient temperature under 50 mbar for 18 hours.

Gas chromatography (GC) is carried out using a Cyclodex B column composed of permethylated β-cyclodextrin deposited in a silicone oil composed of 86% of dimethylsiloxane units and of 14% of methyl(cyano-propyl)siloxane units. The column has a length of 30 m, an internal diameter of 250 μm and a thickness of silicone oil film of 0.25 μm. The diol (compound (I)) is eluted with a relative retention time of 1.00, the R monoacetate (compound (III)) with a relative retention time of 1.10, the S monoacetate (compound (II)) with a relative retention time of 1.13 and the diacetate (compound (IV)) with a relative retention time of 1.38.

The area percentage of the peaks extracted from the chromatograph is subsequently measured for the compounds (I), (II), (III) and (IV). The results appear in table I:

TABLE I

| | Pan- | | | | | | | |
|---|---|---|---|---|---|---|---|---|
| | creatin | QLG | QLG | QLG | QLG | QLC | QL | L10 |
| Degree of | 20 | 78.9 | 88.6 | 93.5 | 98.5 | 98.7 | 99 | 99 |
| conversion | | | | | | | | |
| of the | | | | | | | | |
| compound | | | | | | | | |
| (I) (%) | | | | | | | | |
| Enantiomeric | 67 | 80 | 74 | 87 | 94 | 94 | 97 | 97 |
| excess of | | | | | | | | |
| compound | | | | | | | | |
| (II) (%) | | | | | | | | |
| Selectivity | 10.3 | 7.1 | 5.6 | 4.8 | 3.9 | 3.9 | 3.3 | 3.5 |
| Yield of | 16.5 | 61.9 | 69.1 | 72.5 | 75.8 | 76.4 | 75.5 | 76.4 |
| compound | | | | | | | | |
| (II) (%) | | | | | | | | |
| Reaction | 6.5 | 6.5 | 6.5 | 6.5 | 6.5 | 6.5 | 6.5 | 6.5 |
| time | | | | | | | | |
| (hours) | | | | | | | | |
| Amount | 100 | 3.6 | 5.3 | 7.0 | 10.0 | 10.0 | 5.0 | 5.0 |
| of enzyme | | | | | | | | |
| (as % by | | | | | | | | |
| weight | | | | | | | | |
| with respect | | | | | | | | |
| to the diol) | | | | | | | | |

The degree of conversion of the compound (I) (%) is calculated in the following way: (area percentage of the compound (I) at time 0 (beginning of the reaction)

the area percentage of the compound (I) at the end of the reaction)/area percentage of the compound (I) at time 0.

The enantiomeric excess of compound (II) (%) is calculated in the following way: (absolute value of the (area percentage of the compound (II)−area percentage of the compound (III)))/(area percentage of the compound (II)+area percentage of the compound (III)).

The selectivity is calculated in the following way: it corresponds to the (area percentage of the compounds (II)+(III))/(area percentage of the compound (IV)).

The yield of compound (II) (%) is calculated in the following way: (area percentage of the compound (II))/(area percentage of the compound (I) at time 0).

It is thus observed that the use of the lipase makes it possible to obtain a transesterification reaction of the diol to produce a compound (II) with a better performance than that obtained with an enzyme of animal origin, with markedly lower amounts.

As a reaction scheme of the type:

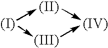

occurs, the low amount of the compound (IV) obtained at the end of the reaction using the enzyme pancreatin leads to a high selectivity measurement.

1. A process for the manufacture of a compound of formula (II): 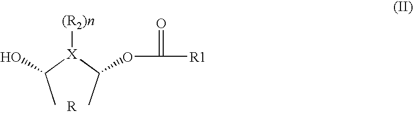
in which:
R is a covalent bond or a hydrocarbon chain comprising having from 1 to 10 carbon atoms;
R1 is a hydrocarbon group comprising from 1 to 10 carbon atoms;
R₂ corresponds to a hydrogen atom and n is an integer between 0 and 2;
X is an atom chosen selected from the group consisting of carbon, nitrogen, oxygen and sulfur;
comprising at least the following stages:
a) reacting at least one compound of formula (I): 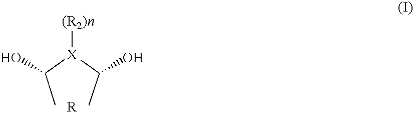
with an acylating agent in an organic solvent in the presence of a lipase of the class EC 3.1.1.3 from Alcaligenes spp., so as to form the corresponding compound of formula (II);
b) isolating the resultant compound of formula (II).

2. The process as claimed in claim 1, wherein the lipase of the class EC 3.1.1.3 exhibits the following characteristics:
an enantiomeric excess of compound (II) of greater than or equal to 50%;
a selectivity for compounds (II) and (III) of greater than or equal to 2, wherein compound (III) has the formula: 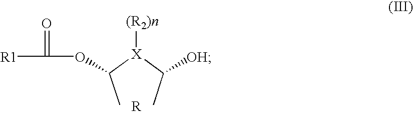
a yield of compound (II) of greater than or equal to 40%; and
a degree of conversion of the compound (I) of greater than or equal to 70%.

3. The process as claimed in claim 1, wherein the lipase is selected from the group consisting of the QL lipase from Alcaligenes sp. PL-266, registered under the number FERM-P No. 3187, and the PL lipase from Alcaligenes sp. PL-679, registered under the number FERM-P No. 3783.

4. The process as claimed in claim 1, wherein the lipase is immobilized on an appropriate solid support.

5. The process as claimed in claim 4, the solid support is selected from the group consisting of DEAE cellulose, DEAE sepharose, diatomaceous earth, silica, alumina, and polypropylene and their mixtures.

6. The process as claimed in claim 1, wherein the lipase is selected from the group consisting of the QL, QLC, QLG, PL, PLC and PLG lipases.

7. The process as claimed in claim 1, wherein R is a hydrocarbon chain having at least one unsaturation.

8. The process as claimed in claim 1, wherein the compound of formula (I) is selected from the group consisting of the compounds of formula (V), (VI) and (VII):

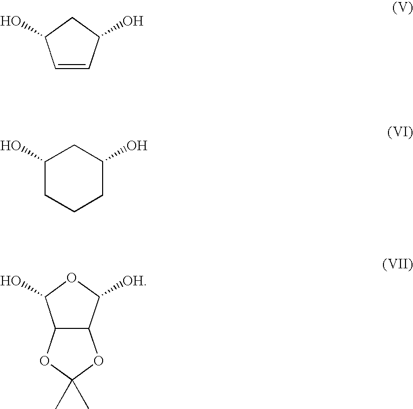

9. The process as claimed in claim 1, wherein the proportion of lipase is between 0.1 and 30% by weight with respect to the weight of the compound of formula (I).

10. The process as claimed in claim 1, wherein the organic solvent is selected from the group consisting of: ketones, ethers, nitriles, and aromatic compounds.

11. The process as claimed in claim 1, wherein the reaction medium of stage a) comprises water.

12. The process as claimed in claim 1, wherein the acylating agent is a compound of formula (VIII):
R1-COO-R₃ (VIII)
in which:
R1 is defined in claim 1; and
R₃ is a hydrocarbon group having from 1 to 10 carbon atoms.

13. The process as claimed in claim 1, wherein the acylating agent is selected from the group consisting of acetates, benzoates and isobutyrates.

14. The process as claimed in claim 1, wherein the acylating agent is selected from the group consisting of vinyl acetate, ethyl acetate, isopropyl acetate, 2,2,2-trifluoroethyl acetate and isopropenyl acetate.

15. The process as claimed in claim 1, wherein the reaction of stage a) is carried out at a temperature of between −5 and 40° C.

16. The process as claimed in claim 1, wherein the duration of the enzymatic reaction of stage a) is between 1 and 24 hours.

17. (canceled)

18. The process as claimed in claim 1, wherein the lipase is not immobilized on an appropriate solid support.

19. The process as claimed in claim 1, wherein the organic solvent is selected from the group consisting of acetone, methyl ethyl ketone, cyclohexanone, cyclopentanone, methyl isobutyl ketone (MIBK), methyl tert-butyl ether (MTBE), tetrahydrofuran (THF), acetonitrile and toluene.
